(19) United States
(12) Patent Application Publication
(10) Pub. No.: US 2017/0264078 A1
(43) Pub. Date: Sep. 14, 2017

(54) ILLUMINATION APPARATUS AND ENDOSCOPE INCLUDING THE ILLUMINATION APPARATUS

(71) Applicant: OLYMPUS CORPORATION, Tokyo (JP)

(72) Inventors: Bakusui DAIDOJI, Hachioji-shi (JP); Eiji YAMAMOTO, Musashimurayama-shi (JP)

(73) Assignee: OLYMPUS CORPORATION, Tokyo (JP)

(21) Appl. No.: 15/605,112

(22) Filed: May 25, 2017

Related U.S. Application Data
(63) Continuation of application No. PCT/JP2014/081248, filed on Nov. 26, 2014.

(51) Int. Cl. H01S 5/06; A61B 1/04; H04N 5/225; G02B 27/48; H01S 5/40; H04N 5/235; A61B 1/06; G02B 23/24

(52) U.S. Cl. CPC H01S 5/0617; A61B 1/0684; A61B 1/04; G02B 23/2469; G02B 27/48; H01S 5/4012; H04N 5/2352; H04N 5/2256; H04N 2005/2255

(57) ABSTRACT

An illumination apparatus includes at least one laser diode, an illumination section, and a light control circuit. The illumination section uses light emitted from the laser diode as illumination light. The light control circuit controls light intensity of the laser diode by pulse-modulating a drive current supplied to the laser diode. The light control circuit controls the light intensity of the laser diode in combination with a duty ratio and a peak current of the pulse-modulated pulse drive current in combination in a multi-oscillation mode region in which a wavelength spectrum width of the light emitted from the laser diode is equal to or larger than a threshold wavelength width.

### CROSS-REFERENCE TO RELATED APPLICATIONS

This application is a Continuation Application of PCT Application No. PCT/JP2014/081248, filed Nov. 26, 2014, the entire contents of all of which are incorporated herein by references.

### BACKGROUND OF THE INVENTION

1. Field of the Invention

The present invention relates to an illumination apparatus that irradiates an observation target with light emitted from a laser diode as illumination light, and an endoscope including the illumination apparatus.

2. Description of the Related Art

In recent years, illumination apparatuses using a semiconductor laser have been actively developed. The illumination apparatuses using a semiconductor laser have the advantages of small size, high brightness and low power consumption, whereas they cause speckles due to high coherence of laser light.

The speckles are interference pattern caused by irradiating an object with light having high coherence such as laser light to reflect the light on the surface of the object and overlap the phases of scattered light, and the interference pattern reflect a state of the vicinity of the surface of the object. Since the speckles cause deterioration of the image quality, technology for reducing the speckles is under development.

The speckle reducing technology is disclosed in, for example, Jpn. Pat. Appln. KOKAI Publication No. 2010-042153. Jpn. Pat. Appln. KOKAI Publication No. 2010-042153 discloses an illumination apparatus that reduces speckles by including a high-frequency superimposing means for superimposing a high-frequency signal on drive current to be supplied to a semiconductor laser to oscillate the semiconductor laser in multi-mode.

### BRIEF SUMMARY OF THE INVENTION

A first illumination apparatus of one embodiment of the invention comprises at least one laser diode, an illumination section which uses light emitted from the laser diode as illumination light, and a light control circuit which controls light intensity of the laser diode by pulse-modulating a drive current supplied to the laser diode, wherein the light control circuit controls the light intensity of the laser diode in combination with a duty ratio and a peak current of the pulse-modulated pulse drive current in a multi-oscillation mode region in which a wavelength spectrum width of the light emitted from the laser diode is equal to or larger than a threshold wavelength width.

A second illumination apparatus of another embodiment of the invention comprises at least one laser diode, an illumination section which uses light emitted from the laser diode as illumination light, and a light control circuit which controls light intensity of the light emitted from the laser diode by pulse-modulating a drive current supplied to the laser diode, wherein the light control circuit controls the light intensity of the laser diode in combination with a duty ratio and a peak current of the pulse-modulated pulse drive current in combination in a speckle reduction region in which a variation in brightness caused when an observation target is irradiated with the illumination light is equal to or smaller than a threshold value.

An endoscope including an illumination apparatus of one embodiment of the invention, the above-mentioned first illumination apparatus, and an image sensor which images an observation target, when the light control circuit sets a frequency of the pulse drive current obtained by the pulse modulation to an integral multiple which is larger than two for a frame rate of the image sensor.

An endoscope including an illumination apparatus of another embodiment of the invention, the above-mentioned second illumination apparatus, and an image sensor which images the observation target, when the light control circuit sets a frequency of the pulse drive current obtained by the pulse modulation to an integral multiple which is larger than two for a frame rate of the image sensor.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWINGS

The accompanying drawings, which are incorporated in and constitute apart of the specification, illustrate embodiments of the invention, and together with the general description given above and the detailed description of the embodiments given below, serve to explain the principles of the invention.

FIG. 1 is a schematic configuration diagram showing an endoscope system to which an illumination apparatus for endoscope according to a first embodiment of the present invention is applied.

FIG. 2 is a block diagram showing an illumination apparatus for endoscope in the endoscope system.

FIG. 3 is a configuration diagram showing an optical diffuser.

FIG. 4 is a chart showing the intensity of laser light emitted from each of first to third LDs relative to the pulse drive current.

FIG. 5 is a schematic diagram showing a multi-oscillation mode region.

FIG. 6 is a chart showing variations in wavelength spectrum width of laser light relative to the peak current of pulse drive current obtained when pulse-amplitude light control is made.

FIG. 7 is a chart showing variations in wavelength spectrum width W of laser light relative to the duty ratio when the peak current of pulse drive current is set to a current value and the duty ratio D is controlled (pulse width light control).

FIG. 8 is a chart showing the minimum light intensity state in a multi-oscillation mode region.

FIG. 9 is a chart showing a route from the maximum light intensity state to the minimum light intensity state in the multi-oscillation mode region when a light control is made mainly by controlling the peak current of pulse drive current by the light control circuit (pulse amplitude light control).

FIG. 10 is a chart showing a route from the maximum light intensity state to the minimum light intensity state in the multi-oscillation mode region when a light control is made mainly by controlling the duty ratio of pulse drive current by the light control circuit (pulse amplitude light control).

FIG. 11 is a schematic diagram showing a function between the light control circuit, input circuit and image processor.

FIG. 12 is a block diagram showing an illumination apparatus for endoscope according to a second variant.

FIG. 13 is a schematic diagram showing a function between the light control circuit, input circuit and image processor in the second variant.

FIG. 14 is a schematic chart showing a speckle reduction region.

FIG. 15 is a chart showing a route from the maximum light intensity state to the minimum light intensity state in the multi-oscillation mode region when the light control is made mainly by controlling the peak current IH of pulse drive current I by the light control circuit (pulse amplitude light control) in the speckle reduction region.

### DETAILED DESCRIPTION OF THE INVENTION

### First Embodiment

An endoscope system including an illumination apparatus according to a first embodiment will be described below with reference to the drawings.

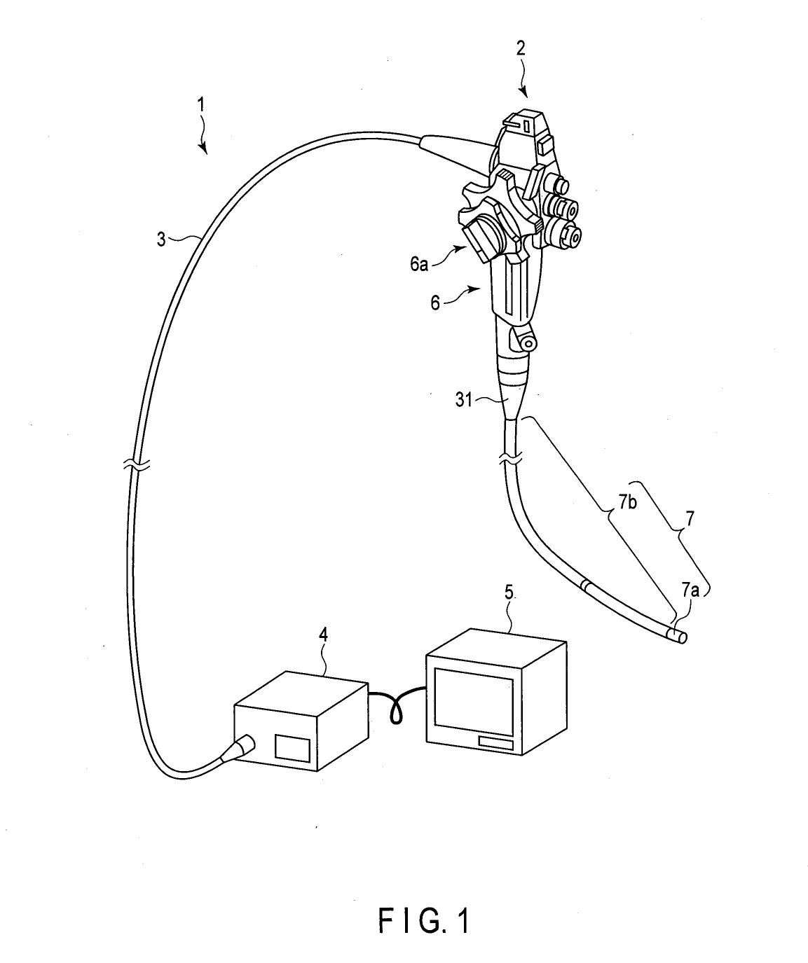

FIG. 1 is a schematic configuration diagram of an endoscope system 1 including an illumination apparatus. The endoscope system 1 includes a scope unit 2, an endoscope main body 4 connected to the scope unit 2 through a cable 3, and an image display 5 connected to the endoscope main body 4. The scope unit 2 will be referred to as the so-called endoscope.

The scope unit 2 includes the cable 3, an operation section 6 and an insertion section 7 coupled to the operation section 6. The operation section 6 includes an operation handle 6a. The operation handle 6a is intended to bend the insertion section 7 in up-and-down directions or right-and-left directions.

The insertion section 7 is intended to observe an object to be observed in an observation target by inserting it into, for example, a tube of the observation target. The insertion section 7 includes an insertion distal-end portion 7a that is formed rigidly and the other portion (referred to as an insertion bending portion hereinafter) 7b that is formed flexibly. Thus, the insertion bending portion 7b is passively bendable and if it is inserted into, for example, a tube of an object to be observed, it is bent along the shape of the tube. The insertion section 7 is also bent in the up and down direction or right and left direction by the operation of the operation section 6.

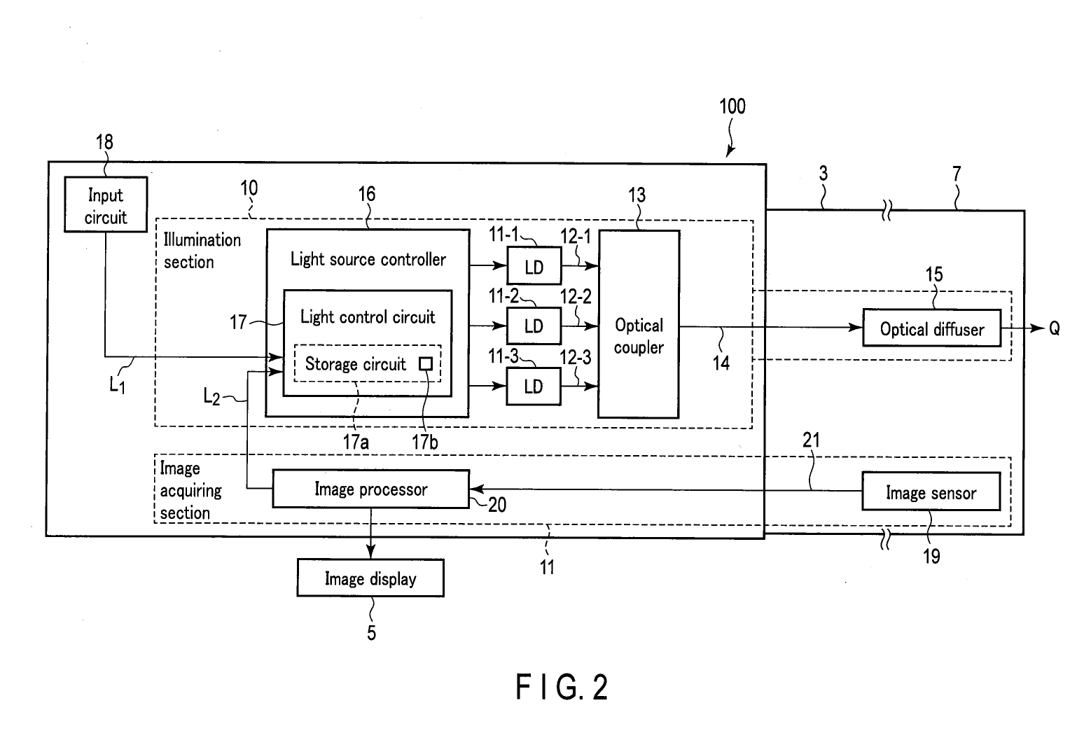

FIG. 2 is a block diagram of an illumination apparatus 100 for endoscope in the endoscope system 1. The endoscope main body 4 includes an illumination section 10 that irradiates an observation target with illumination light and an image acquiring section 11 that acquires an image of the observation target. The image display 5 that displays an image of the observation target is connected to the image acquiring section 11.

The illumination section 10 includes a plurality of laser diodes (referred to as LDs hereinafter), e.g. three first to third LDs 11-1 to 11-3, first to third optical fibers 12-1 to 12-3, an optical coupler (referred to as optical fiber combiner hereinafter) 13, a fourth optical fiber 14, an optical diffuser 15 and a light source controller 16.

The first to third LDs 11-1 to 11-3 oscillate at different oscillation wavelengths and emit laser light. For example, the first LD 11-1 emits blue laser light whose central wavelength is 445 nm, the second LD 11-2 emits green laser light whose central wavelength is 532 nm, and the third LD 11-3 emits red laser light whose central wavelength is 635 nm.

The first optical fiber 12-1 optically connects the first LD 11-1 and the optical coupler 13 and guides blue laser light emitted from the first LD 11-1 to the optical coupler 13.

The second optical fiber 12-2 optically connects the second LD 11-2 and the optical coupler 13 and guides green laser light emitted from the second LD 11-2 to the optical coupler 13.

The third optical fiber 12-3 optically connects the first LD 11-3 and the optical coupler 13 and guides red laser light emitted from the third LD 11-3 to the optical coupler 13.

The optical fiber combiner 13 combines the blue laser light, green laser light and red laser light guided by the first optical fiber 12-1, second optical fiber 12-2 and third optical fiber 12-3, respectively into white laser light.

The fourth optical fiber 14 guides the white laser light combined by the optical fiber combiner 13 to the optical diffuser 15.

The first to third optical fibers 12-1 to 12-3 and the fourth optical fiber 14 are each a single fiber whose core diameter is, for example, several tens of μm to several hundreds of μm.

Coupling lenses (not shown) are provided between the first to third optical fibers 12-1 to 12-3 and the fourth optical fiber 12-4. The coupling lenses cause blue laser light, green laser light and red laser light emitted from the first to third optical fibers 12-1 to 12-3 to converge, and couple them to the fourth optical fiber 12-4.

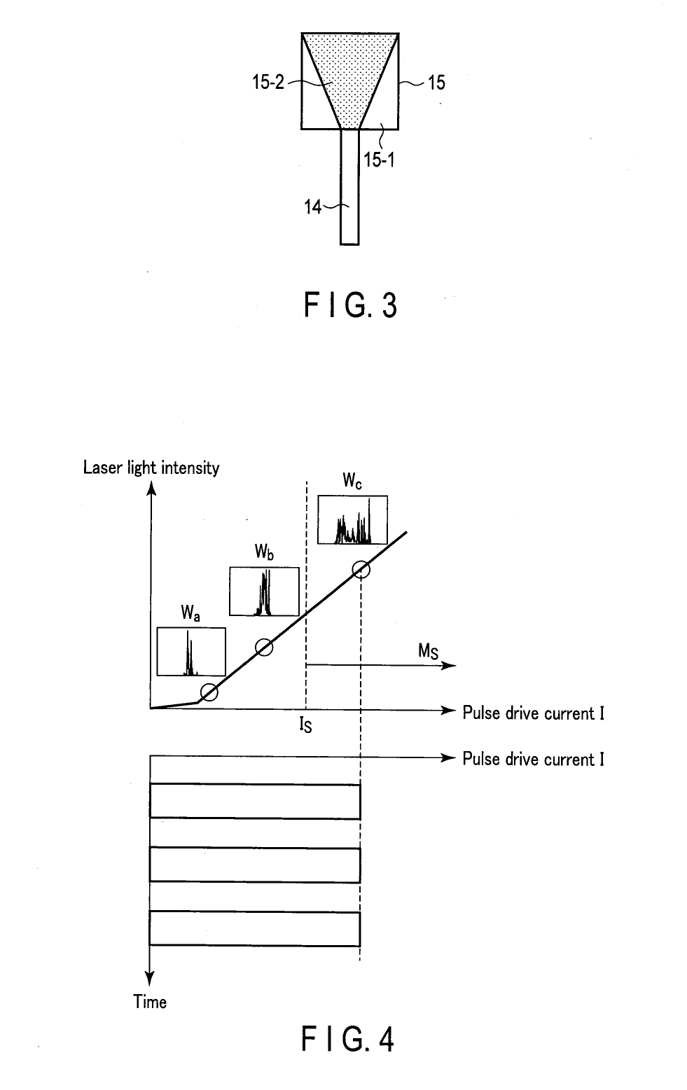

FIG. 3 is a configuration diagram of the optical diffuser 15. The optical diffuser 15 diffuses the white laser light guided by the fourth optical fiber 14. The white laser light diffused by the optical diffuser 15 is emitted as illumination light Q. The optical diffuser 15 includes a holder 15-1 and a diffusion member 15-2 such as an alumina particle, which is contained in the holder 15-1. The light diffusion of the optical diffuser 15 brings about the advantage of widening the distribution of the white laser light guided by the fourth optical fiber 14 and disturbing the phase of the white laser beam to reduce the coherence and reduce the speckles.

The light source controller 16 includes a light control circuit 17 to control light intensity for the first to third LDs 11-1 to 11-3. The light control circuit 17 turns on and off the first to third LDs 11-1 to 11-3 and controls light intensity for the first to third LDs 11-1 to 11-3. In the light control, pulse drive currents I supplied to the first to third LDs 11-1 to 11-3 are independently pulse-modulated, respectively.

If the wavelength spectrum width of each of the blue laser light, green laser light and red laser light emitted from the first to third LDs 11-1 to 11-3 is not less than a threshold wavelength width, they will fall within a multi-oscillation mode region Ms as shown in FIG. 4.

The light control circuit 17 pulse-modulates the first to third LDs 11-1 to 11-3 in combination with the control of peak current IH of pulse drive current I obtained by pulse modulation (pulse-amplitude light control) and the control of duty ratio D of the pulse drive current I (pulse-width light control) in the multi-oscillation mode region Ms of the first to third LDs 11-1 to 11-3.

Specifically, the light control circuit 17 includes a storage circuit 17a. In the storage circuit 17a, a light control table 17b is contained. The light control table 17b stores light control information about setting of duty ratio D and peak current IH of pulse drive current I in the multi-oscillation mode region Ms.

The storage circuit 17a stores information indicating the ratio of intensities of blue, green and red laser light emitted from the first to third LDs 11-1 to 11-3 such that illumination light Q has a desired color (referred to as light intensity ratio information hereinafter). The desired color is, for example, white light with high color rendering properties, or the color of illumination light Q to reproduce the color of an observation target which is irradiated with light emitted from, e.g. a xenon lamp or a halogen lamp. The information recorded in the storage circuit 17a will be described in detail later.

An input circuit 18 and image acquiring section 11 are connected to the light control circuit 17. The light control circuit 17 is supplied with first light intensity control information L1 for illumination light Q output from the input circuit 18 or second light intensity control information L2 output from the image acquiring section 11. The first light intensity control information L1 is information to cause the image of an observation target to have an appropriate brightness value. The appropriate brightness value is the one having an appropriate brightness to prevent halation and black defects from being caused on the image of an observation target. The second light intensity control information L2 is information to cause the image of an observation target to have an appropriate brightness value.

The light control circuit 17 controls light intensity of the first to third LDs 11-1 to 11-3 in combination with the control of duty ratio D and that of peak current IH for pulse drive current I supplied to the first to third LDs 11-1 to 11-3 on the basis of the first light intensity control information L1 or the second light intensity control information L2.

FIG. 4 shows light intensity F of blue, green and red laser light emitted from the first to third LDs 11-1 to 11-3 relative to the pulse drive current I.

In the pulse modulation, illumination light Q of laser light intensity F corresponding to the pulse drive current I is emitted as shown in FIG. 4. Though FIG. 4 shows laser light intensity F relative to the pulse drive current I of one LD, the same holds true for the first to third LDs 11-1 to 11-3.

If the peak current of pulse drive current I increases, the oscillation modes increase and accordingly the wavelength spectrum width W (Wa<Wb<Wc) becomes large. The wavelength spectrum widths Wa, Wb and Wc are each defined by, for example, a wavelength width to halve the intensity relative to the peak intensity of the wavelength spectrum.

The reason that the oscillation modes increase is as follows. If the pulse drive current I supplied to the first to third LDs 11-1 to 11-3 increases, the carrier density and refractive index in each of the LDs 11-1 to 11-3 vary. If the intensity F of laser light emitted from the first to third LDs 11-1 to 11-3 increases, the carrier density and refractive index also vary to increase the oscillation modes due to the rise of the internal temperature of the LDs 11-1 to 11-3.

The duty ratio D of pulse drive current I is the proportion of light-emission time (=heat-generation time) of the first to third LDs 11-1 to 11-3 to the light-out time (=cooling time) thereof (light-emission time/light-out time). If the duty ratio D increases, the light-emission time (=heat-generation time) of each of the first to third LDs 11-1 to 11-3 is lengthened and thus the internal temperature of the first to third LDs 11-1 to 11-3 increases.

As described above, the oscillation modes increase as the internal temperature of the first to third LDs 11-1 to 11-3 increases. If, therefore, the duty ratio D is increased to a high duty ratio from a low duty ratio, the oscillation modes increase and the wavelength spectrum width W (Wa<Wb<Wc) becomes large.

If the oscillation modes increase and the wavelength spectrum width W (Wa<Wb<Wc) becomes large, temporal coherence lowers, or coherence lowers. Accordingly, the speckles are reduced.

When the first to third LDs 11-1 to 11-3 are pulse-modulated, the light control circuit 17 controls the duty ratio D and the peak current IH for the pulse drive current I within the multi-oscillation mode region Ms in which the wavelength spectrum width W (Wa, Wb, Wc) of laser light emitted from the first to third LDs 11-1 to 11-3 becomes not smaller than the threshold wavelength width. In other words, if the peak current IH of pulse drive current I becomes not smaller than multi-oscillation mode threshold current Is as shown in FIG. 4, the first to third LDs 11-1 to 11-3 will fall within the multi-oscillation mode region Ms. In the multi-oscillation mode region Ms, the light control circuit 17 controls the duty ratio D and the peak current IH relative to the pulse drive current I.

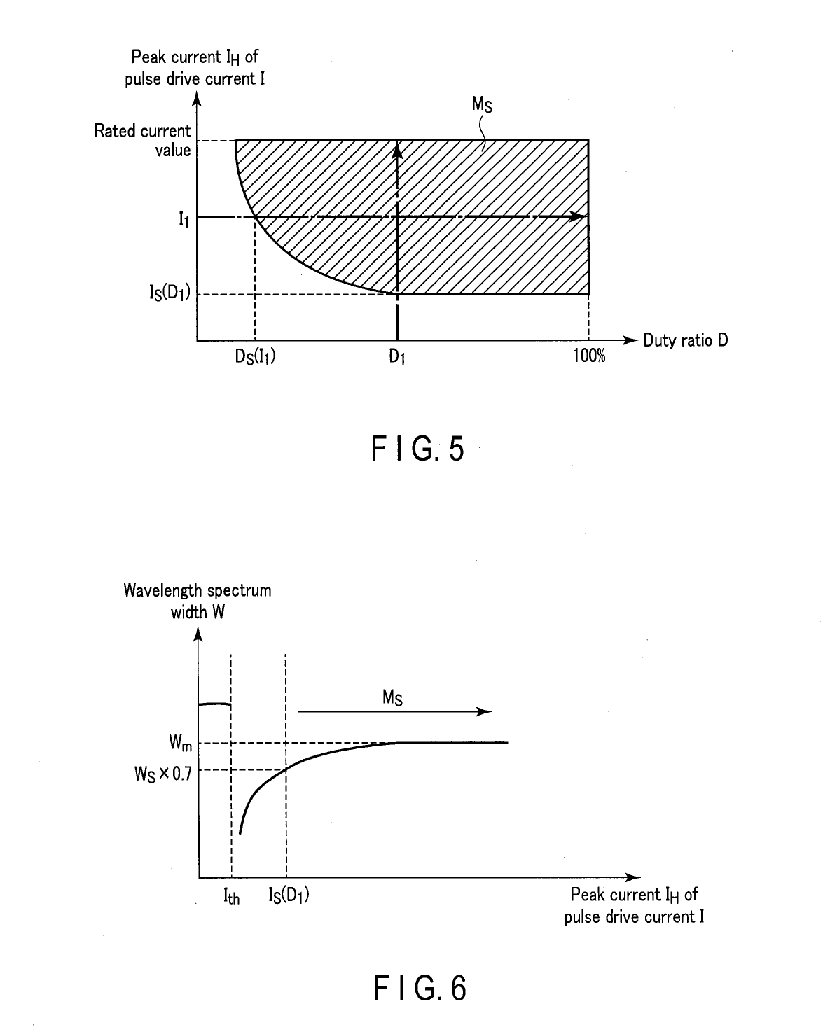

The multi-oscillation mode region Ms of a single LD, or one of the first to third LDs 11-1 to 11-3 here will be described with reference to the schematic diagram of multi-oscillation mode region Ms shown in FIG. 5.

The multi-oscillation mode region Ms occurs a region where depends upon the relationship between the duty ratio D and the peak current IH of pulse drive current I.

In the multi-oscillation mode region Ms, the duty ratio D when the wavelength spectrum width W becomes 70% of the maximum wavelength spectrum width will be referred to as a multi-oscillation mode threshold duty ratio Ds.

The peak current IH of the pulse drive current when the wavelength spectrum width W becomes 70% of the maximum wavelength spectrum width will be referred to as a multi-oscillation mode threshold current Is.

If, therefore, the duty ratio D is equal to or larger than the multi-oscillation mode threshold duty ratio Ds and the peak current IH of the pulse drive current is equal to or larger than multi-oscillation mode threshold current Is, one of the first to third LDs 11-1 to 11-3 will fall within the multi-oscillation mode region Ms.

FIG. 6 shows variations in the wavelength spectrum width of laser light relative to the peak current IH of pulse drive current I obtained when pulse-amplitude light control is made.

A threshold wavelength width Ws to determine the multi-oscillation mode region Ms is set to 70% of the maximum wavelength spectrum width Wm (Wm×0.7) in one of the first to third LDs 11-1 to 11-3 when illumination light Q emitted from the illumination apparatus 100 is in the maximum light intensity state.

Usually, the wavelength spectrum width W becomes the largest in the maximum light intensity state. If it is equal to or larger than the maximum wavelength spectrum width Wm, the speckles are reduced with coherence lowered sufficiently.

If the peak current IH of pulse drive current I increases, the oscillation modes increase and the wavelength spectrum width W (Wa<Wb<Wc) becomes large. When the peak current IH of pulse drive current I is equal to or larger than a current value, the oscillation modes do not increase but the wavelength spectrum width W is saturated. When saturated, the wavelength spectrum width W becomes equal to the maximum wavelength spectrum width Wm.

The peak current IH of pulse drive current I when the wavelength spectrum width W is 70% of the maximum wavelength spectrum width Wm is defined as a multi-oscillation mode threshold current Is as described above. A region whose current is equal to or larger than the multi-oscillation mode threshold current Is becomes the multi-oscillation mode region Ms. The multi-oscillation mode threshold current Is depends upon the duty ratio D.

In other words, the minimum peak current included in the multi-oscillation mode region Ms relative to the set duty ratio D is defined as a multi-oscillation mode threshold peak current. The light control circuit 17 controls light intensity by controlling the peak current IH of pulse drive current I within a range of not smaller than the multi-oscillation mode threshold current Is. As compared with the duty ratio D to be set, a duty ratio D having no peak current included in the multi-oscillation mode region Ms is not set.

In the first to third LDs 11-1 to 11-3, a lasing threshold current Ith is referred to as the peak current IH of pulse drive current I when the peak current IH increases and stably oscillates laser. In pulse drive current I that is equal to or smaller than the lasing threshold current Ith, the first to third LDs 11-1 to 11-3 increases the wavelength spectrum width W for the light-emission state of an LED that does not oscillate laser. In a region of the peak current IH that is larger than the lasing threshold current Ith, the first to third LDs 11-1 to 11-3 oscillate laser to narrow the wavelength spectrum width W. Thus, the bottom current of pulse drive current I is set to a value that is equal to or smaller than the lasing threshold current Ith.

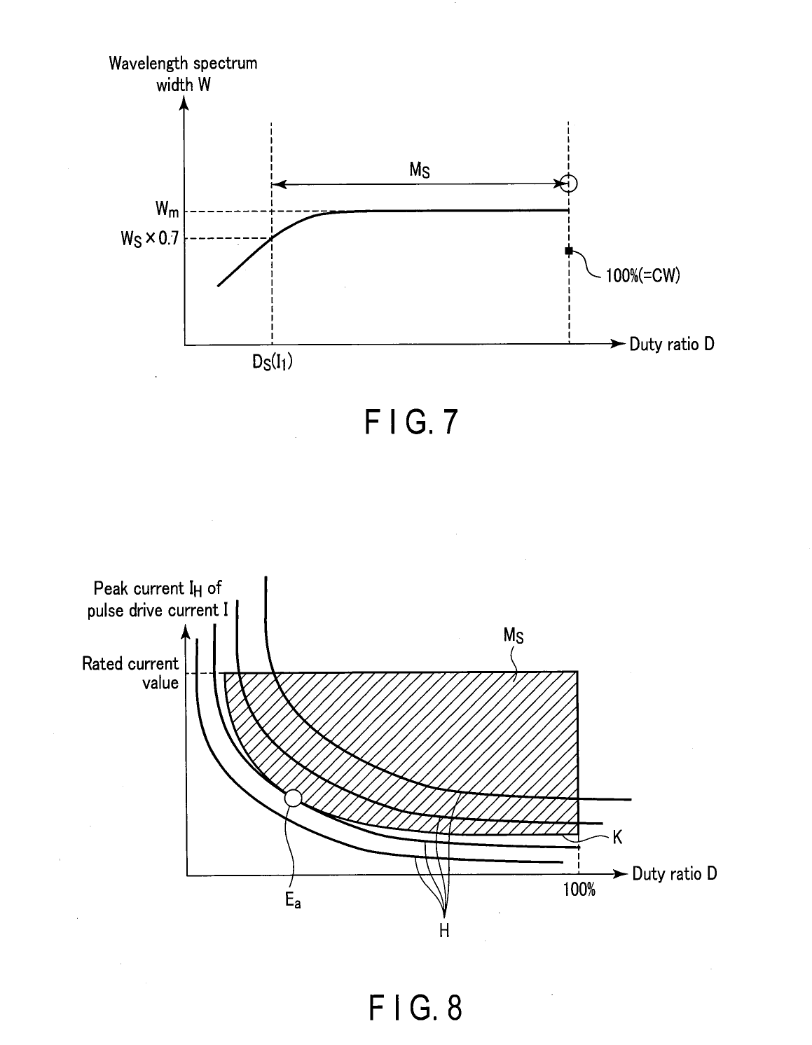

FIG. 7 shows variations in the wavelength spectrum width W of laser light relative to the duty ratio D when the peak current IH of pulse drive current I is set to a current value I1 and the duty ratio D of pulse drive current I is controlled (pulse width light control).

Since the duty ratio D is the proportion of light-emission time (=heat-generation time) to the light-out time (=cooling time), if the duty ratio D increases, the temperature in the elements of the first to third LDs 11-1 to 11-3 increases. As the temperature increases, the first to third LDs 11-1 to 11-3 increase in the oscillation modes. Like the above, therefore, if the duty ratio D increases to a high duty ratio from a low duty ratio and becomes not lower than a certain duty ratio D, the oscillation modes do not increase but the wavelength spectrum width W is saturated. Then, the wavelength spectrum width W becomes equal to the maximum wavelength spectrum width Wm.

The duty ratio D when the wavelength spectrum width W becomes 70% of the maximum wavelength spectrum width Wm, will be referred to as a multi-oscillation mode threshold duty ratio Ds. A region of the duty ratio D that is not lower than the multi-oscillation mode threshold duty ratio Ds becomes the multi-oscillation mode region Ms.

The light control circuit 17 makes light control by controlling the duty ratio D within a range of not lower than the multi-oscillation mode threshold duty ratio Ds.

The carrier density and refractive index when the light intensity of the first to third LDs 11-1 to 11-3 is controlled by pulse-modulating the pulse drive current I vary more greatly than when it is controlled by supplying the pulse drive current I continuously (CW: duty ratio D is 100%). At the time of pulse modulation, therefore, the wavelength spectrum width W becomes larger than at the time of CW (duty ratio D is 100%). Thus, the state at the time of CW (duty ratio D is 100%) is not used for light control, and the light control circuit 17 makes light control by controlling the duty ratio D within a range of not lower than the multi-oscillation mode threshold duty ratio Ds and smaller than 100%.

The light control circuit 17 sets the frequency of pulse drive current I obtained by the pulse modulation to an integral multiple n (integer of two or more) which is larger than two for the frame rate of an image sensor 19. The frame rate is, for example, a frequency of 30 Hz (fps). Accordingly, the frequency of the pulse modulation becomes 30×n (Hz).

In the pulse modulation, different laser light oscillation modes are set for pulse drive currents I of different frequencies. If, therefore, the frequency of pulse drive current I is set higher than the frame rate of the image sensor 19, the speckles are averaged in terms of time within exposure time of the image sensor 19 and thus can be reduced.

Since the integral multiple n is the integer of two or more, the intensities of light exposed in the frames of the image sensor 19 become equal. It is thus possible to prevent a flicker due to variations in brightness of moving images acquired by imaging of the image sensor 19.

To average the speckles sufficiently and reduce them effectively, it is favorable that the integral multiple n is 10 or more and it is more favorable that it is 100 or more. Further, when the frequency of pulse drive current I is in a range of MHz or higher, the carrier density and refractive index vary more greatly and the wavelength spectrum width becomes larger, with the result that a greater speckle reduction effect can be obtained.

The foregoing descriptions are directed to the multi-oscillation mode region Ms chiefly for a single LD. For the first to third LDs 11-1 to 11-3, the light control circuit 17 controls the duty ratio D and the peak current IH of pulse drive current I in accordance with light intensity ratio information stored in the storage circuit 17a.

As described above, the storage circuit 17a stores the light intensity ratio information of the first to third LDs 11-1 to 11-3. The light intensity ratio information is calculated based on, e.g. the color temperature and average color rendering index of illumination light Q.

When the light intensity ratio information is determined, the light control circuit 17 sets the intensities of light of the first to third LDs 11-1 to 11-3 on the basis of the light intensity ratio information, first light intensity control information L1 input from the input circuit 18 and second light intensity control information L2 input from an image processor 20.

In the storage circuit 17a, the light control table 17b is contained, as described above. The light control table 17b stores light control information about setting of the duty ratio D and peak current IH of pulse drive current I in the multi-oscillation mode region Ms. The light control information includes information indicating the set light intensity of the first to third LDs 11-1 to 11-3 relative to the first or second light intensity control information L1 or L2, which is set based on the light intensity ratio information, and the relationship in setting between a value of the duty ratio D and that of pulse drive current I relative to the set light intensity.

Creation of the light control table 17b will be described.

Wavelength spectrum width W obtained when the duty ratio D and the peak current IH of pulse drive current I are varied in advance for the first to third LDs 11-1 to 11-3, is measured. Thus, the multi-oscillation mode region Ms can be grasped from the relationship between the duty ratio D and the peak current IH of pulse drive current I as shown in FIG. 5.

In the multi-oscillation mode region Ms, a product (=intensity of emitted light) of the duty ratio Ds and the peak current IH of pulse drive current I is obtained. The minimum light intensity state in which the product of the duty ratio Ds and the peak current IH becomes the smallest and the maximum light intensity state in which the product becomes the largest are obtained, and a light intensity range is set by these minimum and maximum light intensity states.

The minimum light intensity state Ea is represented by a point at which an equal emitted-light intensity curve H and a borderline K of the multi-oscillation mode region Ms in which the wavelength spectrum width W is equal to the threshold wavelength width Ws, are tangent to each other, as shown in FIG. 8. In the maximum light intensity state Ea, for example, the peak current IH of pulse drive current I is the rated current of the first to third LDs 11-1 to 11-3 and the duty ratio D is 99.%. The minimum light intensity state Ea depends upon the multi-oscillation mode region Ms of the first to third LDs 11-1 to 11-3.

When light control is made by combining the control of the duty ratio D and that of the peak current IH for pulse drive current I, a route between the maximum light intensity state Ea and the minimum light intensity state is set to make the light intensity linear.

If a route is set between the maximum light intensity state Ea and the minimum light intensity state, the duty ratio D and the peak current IH of pulse drive current I are assigned to the set light intensity of each of the first to third LDs 11-1 to 11-3. Accordingly, the light control table 17b is created.

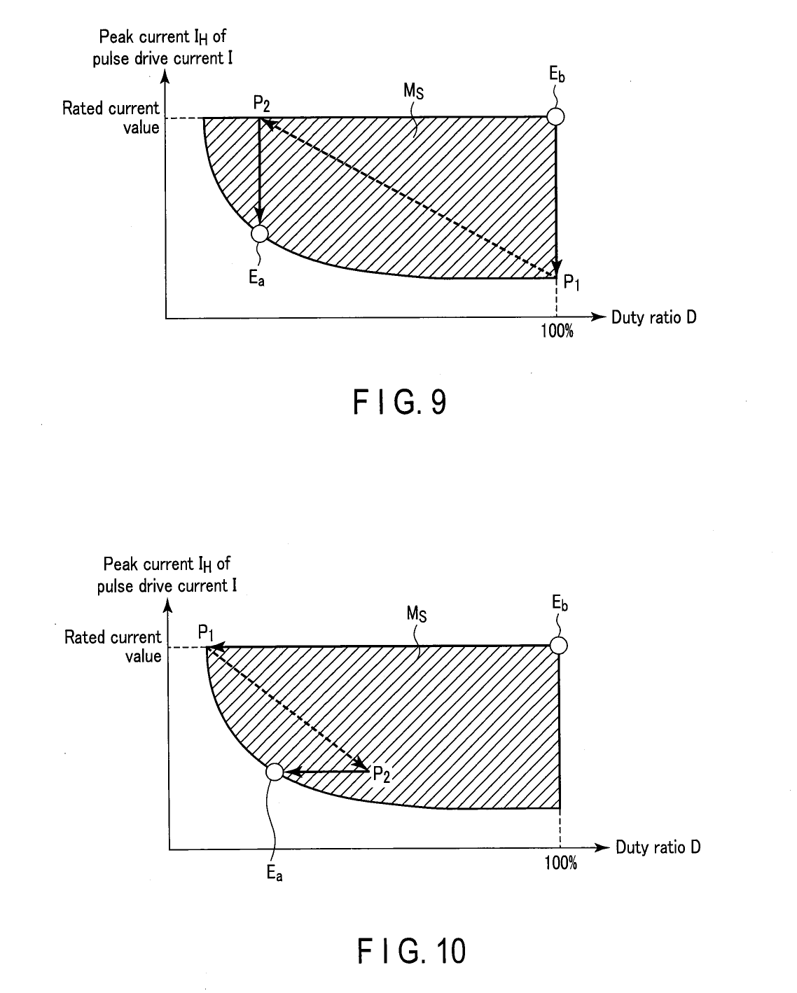

FIG. 9 shows a route from the maximum light intensity state Eb to the minimum light intensity state Ea in the multi-oscillation mode region Ms when the light control is made mainly by controlling the peak current IH of pulse drive current I by the light control circuit 17 (pulse amplitude light control).

In this route of light control, first, light control is made by controlling the peak current IH of pulse drive current I (pulse amplitude light control) from the maximum light intensity state Eb to the multi-oscillation mode threshold current Is (P1 state) in which the duty ratio D is 99%.

Next, in the duty ratio D of the minimum light intensity state Ea, the peak current IH of pulse drive current I is set (P2 state) such that the light intensity is the same as that in the P1 state.

Next, the duty ratio D of pulse drive current I is controlled (pulse amplitude light control) from the P2 state to the minimum light intensity state Ea.

FIG. 10 shows a route from the maximum light intensity state Eb to the minimum light intensity state Ea in the multi-oscillation mode region Ms when light control is made mainly by controlling the duty ratio D of pulse drive current I (pulse amplitude light control).

In this route, light control is made by controlling the duty ratio D of pulse drive current I (pulse amplitude light control) from the maximum light intensity state Eb to the multi-oscillation mode threshold duty ratio Ds (P1 state) at the rated current value.

Next, in the peak current IH of pulse drive current I in the minimum light intensity state Ea, the duty ratio D is set (P2 state) such that the light intensity is the same as that in the P1 state.

Next, the duty ratio D of pulse drive current I is controlled (pulse amplitude light control) from the P2 state to the minimum light intensity state Ea.

In this way, the light control is made mainly by controlling the peak current IH of pulse drive current I (pulse amplitude light control) or controlling the duty ratio D (pulse amplitude light control) in the multi-oscillation mode region Ms. Thus, the light control can be made within a broad variable range with speckles reduced and moreover the light control for the first to third LDs 11-1 to 11-3 can be controlled simply and easily.

In the foregoing routes, the peak current IH is mainly controlled (pulse amplitude light control) when it is not smaller than the multi-oscillation mode threshold current Is or the duty ratio D is mainly controlled (pulse amplitude light control) when it is not lower than the multi-oscillation mode threshold duty ratio Ds. However, not only the routes but also a route to control the peak current IH and the duty ratio D at the same time can be used and, in this case, the route is oblique with respect to the axis of the peak current IH or the duty ratio D.

The image acquiring section 11 includes the image sensor 19 and the image processor 20. The image sensor 19 and image processor 20 are connected through an imaging cable 21. The image sensor 19 receives a light image reflected from an observation target, images the observation target and outputs an imaging signal. Specifically, the image sensor 19 includes, e.g. a CCD imager, a CMOS imager. The frame rate of the image sensor 19 is, e.g. a frequency of 30 Hz (fps).

The image processor 20 receives an image signal from the image sensor 19 and processes the image signal to acquire an image of the observation target. The image processor 20 performs image processing on the basis of brightness information included in the image signal output from the image sensor 19 to calculate second light intensity control information L2. The second light intensity control information L2 is intended to cause the image of the observation target to have an appropriate brightness value and is sent to the light control circuit 17.

The image display 5 displays the image of the observation target which is acquired by the image processor 20. The image display 5 includes a monitor such as a liquid crystal display.

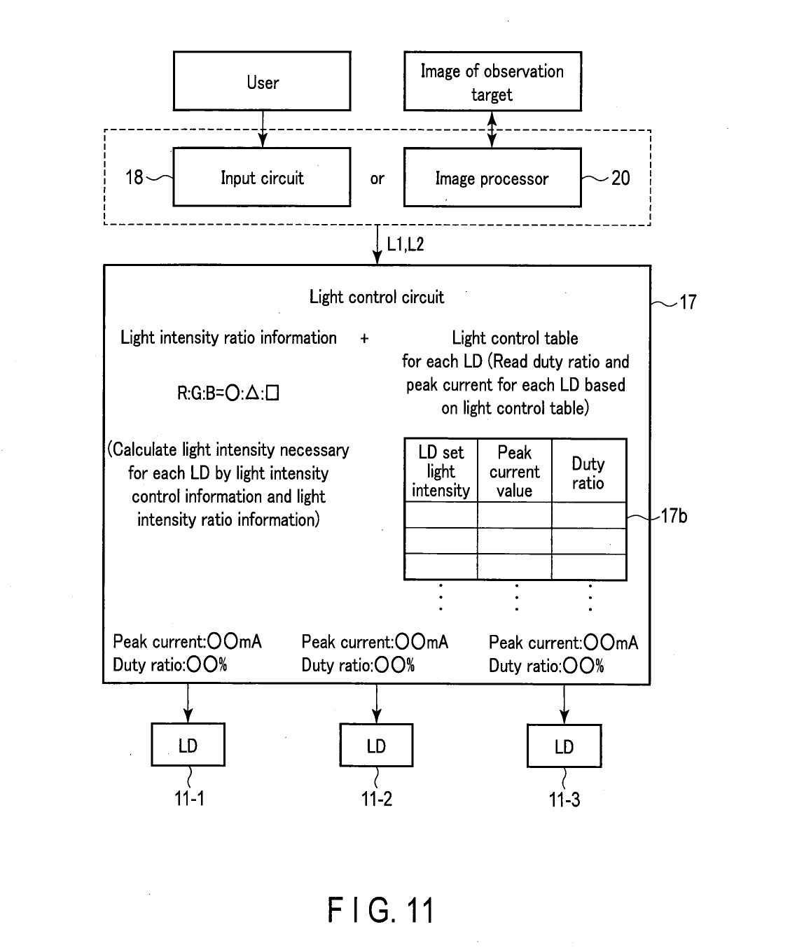

An operation of the illumination apparatus 100 for endoscope that is configured as described above will be described below with reference to the schematic diagram of FIG. 11 showing a function between the light control circuit 17, input circuit 18 and image processor 20.

The input circuit 18 receives an operator's operation and outputs first light intensity control information L1 for illumination light Q.

The image processor 20 performs image processing on the basis of bright information included in the image signal output from the image sensor 19 to calculate second light intensity control information L2. The second light intensity control information L2 is intended to cause the image of an observation target to have an appropriate brightness value and is sent to the light control circuit 17.

The light control circuit 17 controls light intensity of the first to third LDs 11-1 to 11-3 in combination with the control of the duty ratio D and that of peak current IH for pulse drive current I supplied to the first to third LDs 11-1 to 11-3 on the basis of the first light intensity control information L1 or the second light intensity control information L2.

In this case, the light control circuit 17 controls light intensity of the first to third LDs 11-1 to 11-3 in combination with the control of the duty ratio D and that of the peak current IH for pulse drive current I in accordance with light control information stored in the light control table 17b of the storage circuit 17a.

The light control information includes information indicating a set light intensity of the first to third LDs 11-1 to 11-3 for the first or second light intensity control information L1 or L2 and the relationship in setting between a value of the duty ratio D and that of the peak current IH for pulse drive current I for the set light intensity, based on light intensity ratio information indicating the intensity ratio of blue laser light, green laser light and red laser light emitted from the first to third LDs 11-1 to 11-3 to cause illumination light Q to have a desired color.

The first to third LDs 11-1 to 11-3 whose light intensity is controlled emit blue laser light, green laser light and red laser light. These blue, green and red laser lights are guided by their respective optical fibers 12-1, 12-2 and 12-3 and enter the optical fiber combiner 13. The optical fiber combiner 13 combines the blue, green and red laser lights and emits white laser light. The white laser light emitted from the optical fiber combiner 13 is guided by the optical fiber 14 and enters the optical diffuser 15.

The optical diffuser 15 diffuses the white laser light guided by the fourth optical fiber 14. The diffused white laser light is radiated to an observation target as illumination light Q.

The image sensor 19 receives light reflected from the observation target, images the observation target and then outputs an imaging signal.

The image processor 20 receives the image signal from the image sensor 19 and processes the image signal to acquire an image of the observation target. The image of the observation target is displayed on the image display 5.

The image processor 20 performs image processing on the basis of bright information included in the image signal output from the image sensor 19 to calculate second light intensity control information L2. The second light intensity control information L2 is sent to the light control circuit 17.

As described above, according to the first embodiment, light control is made for the first to third LDs 11-1 to 11-3 by combining the control of the duty ratio D and that of the peak current IH for pulse drive current I supplied to the first to third LDs 11-1 to 11-3 in the multi-oscillation mode region Ms. Thus, the light control can be made within a broad variable range with speckles reduced.

The frequency of pulse drive current I is set to an integral multiple n (integer of two or more) which is larger than two for the frame rate of the image sensor 19 to make the frequency of pulse drive current I higher than the frame rate of the image sensor 19. Therefore, the speckles can be averaged in terms of time within exposure time of the image sensor 19 and thus can be reduced.

Since the integral multiple n is the integer of two or more, the intensities of light exposed in the frames of the image sensor 19 become equal. It is thus possible to prevent a flicker due to variations in brightness of moving images acquired by imaging of the image sensor 19.

The speckles can be sufficiently averaged and effectively reduced if favorably the integral multiple n is set to 10 and more favorably it is set to 100 or more.

Furthermore, when the frequency of pulse drive current I is in a range of MHz or higher, the carrier density and refractive index vary more greatly and the wavelength spectrum width becomes larger, with the result that a greater speckle reduction effect can be obtained.

[First Variant]

In the foregoing first embodiment, an observation target is observed by emitting white illumination light Q from the three LDs 11-1 to 11-3. Instead of the three LDs, four or more LDs can be used. If four or more LDs are used, for example, an observation target can be observed using white light with higher color rendering properties than using three LDs.

In the foregoing first embodiment, furthermore, two LDs of a blue-violet LD that emits blue-violet laser light and a green LD that emits green laser light can be added. The use of the two LDs makes it possible to make an observation such as emphatically displaying a blood vessel using light absorption characteristics of hemoglobin.

In the first embodiment, an LD that emits laser light having a near-infrared wavelength can be used for observation.

[Second Variant]

A second variant will be described below. The same elements as those shown in FIG. 2 are denoted by the same reference numeral and their detailed descriptions will be omitted.

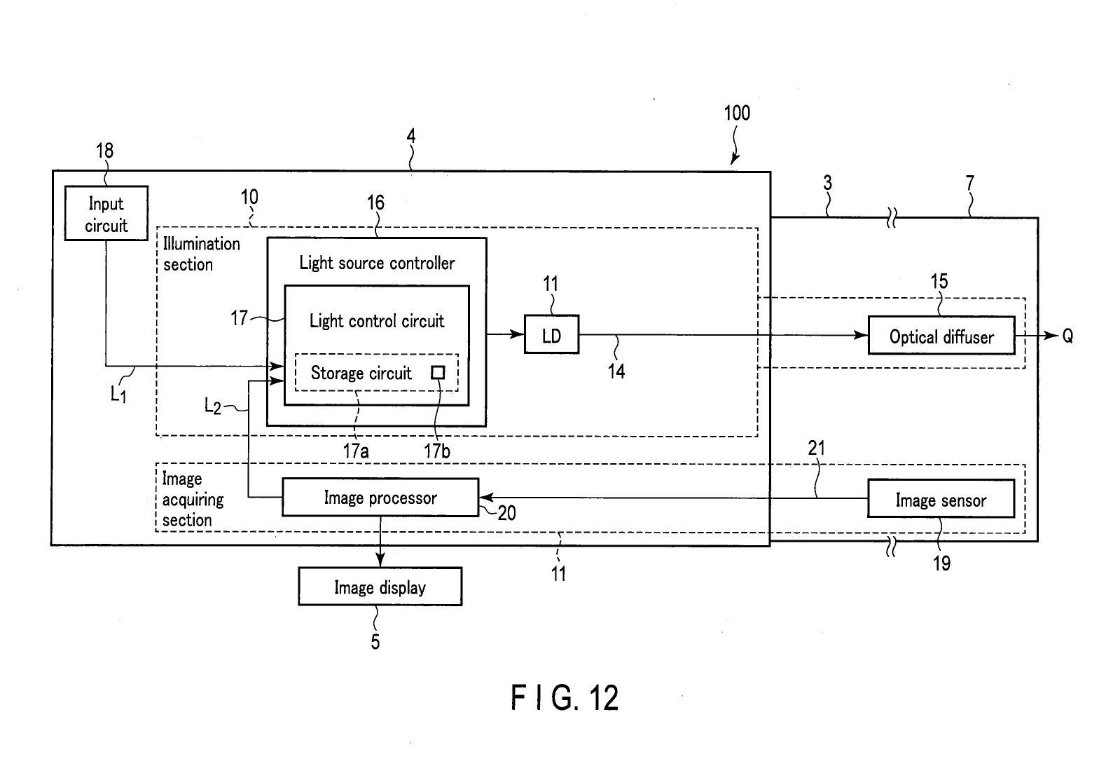

FIG. 12 is a block diagram showing an illumination apparatus 100 for endoscope according to the second variant.

The illumination apparatus 100 includes one LD 11. The LD 11 is, for example, an LD 11-1 that emits blue laser light. If the LD 11-1 is used, the optical diffuser 15 is able to emit white light using a fluorescent material excited by the blue laser light. As the LD 11, for example, an LD that emits laser light having a near-infrared wavelength can be used and an LD that emits laser light having a central wavelength can be used.

The LD 11 is optically connected to the optical diffuser 15 through the optical fiber 14. Since one LD 11 is provided, the need for the optical fiber combiner 13 of the foregoing first embodiment is obviated.

The light control circuit 17 controls light intensity of the LD 11 in combination with the control of the duty ratio D and that of the peak current IH for pulse drive current I supplied to the LD 11 on the basis of the first light intensity control information L1 or the second light intensity control information L2. The light control circuit 17 controls light intensity of the LD 11 in combination with the control of the duty ratio D and that of the peak current IH for pulse drive current I in accordance with light control information stored in the light control table 17b of the storage circuit 17a.

The light control information includes information indicating the set light intensity of the LD 11 relative to the first or second light intensity control information L1 or L2, and the relationship in setting between a value of the duty ratio D and that of pulse drive current I relative to the set light intensity.

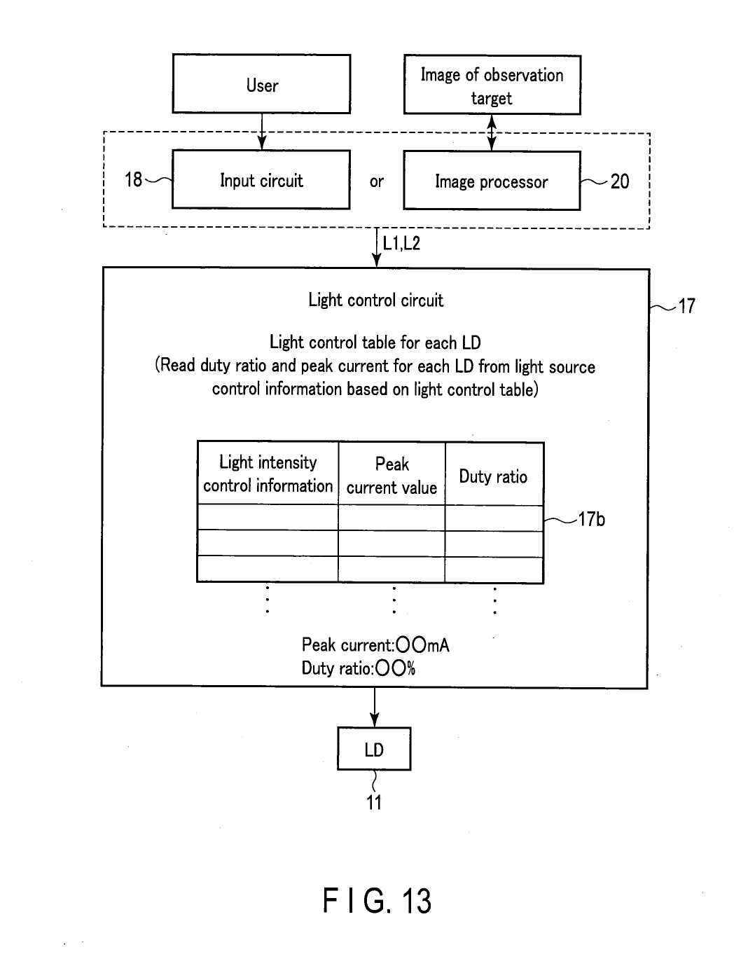

An operation of the illumination apparatus 100 configured as described above, which differs from that in the foregoing first embodiment, will be described with reference to the schematic view of FIG. 13 showing the functions of the light control circuit 17, input circuit 18 and image processor 20.

The light control circuit 17 controls light intensity of the LD 11 in combination with the control of the duty ratio D and that of the peak current IH for pulse drive current I supplied to the LD 11 on the basis of the first light intensity control information L1 or the second light intensity control information L2. The light control circuit 17 controls light intensity of the first to third LDs 11-1 to 11-3 in combination with the control of the duty ratio D and that of the peak current IH for pulse drive current I in accordance with light control information stored in the light control table 17b of the storage circuit 17a. The light control information includes information indicating the set light intensity of the LD 11 relative to the first or second light intensity control information L1 or L2, and the relationship in setting between a value of the duty ratio D and that of pulse drive current I relative to the set light intensity.

The LD 11 emits, for example, blue laser light. The laser light is guided by the optical fiber 14 and enters the optical diffuser 15. The optical diffuser 15 diffuses the laser light guided by the optical fiber 14 and at the same time emits fluorescence excited by irradiation of the blue laser light. The diffused blue laser light and the fluorescence are radiated to an observation target as illumination light Q.

As described above, according to the second variant, light control is made for the LD 11 in combination with the control of the duty ratio D and that of the peak current IH for pulse drive current I supplied to the LD 11 in the multi-oscillation mode region Ms. Thus, the same advantage as that of the first embodiment can be obtained.

### Second Embodiment

An illumination apparatus for endoscope according to a second embodiment of the present invention will be described below.

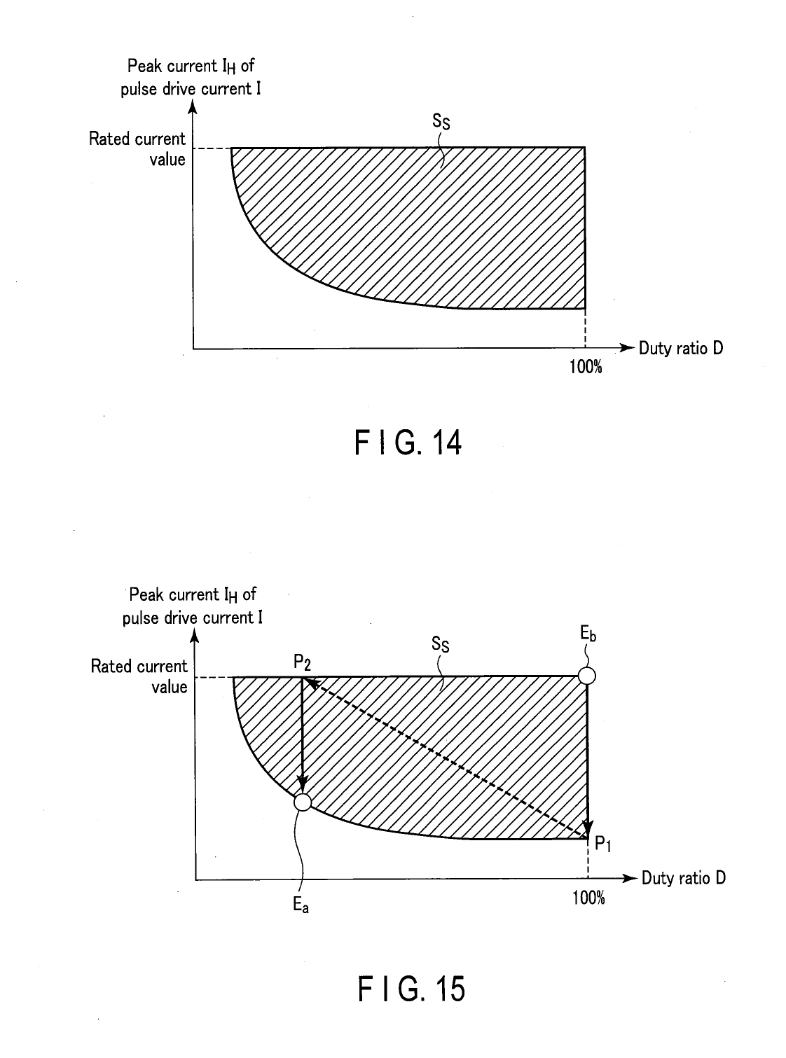

In the second embodiment, the light control circuit 17 controls light intensity of the first to third LDs 11-1 to 11-3 or the LD 11 in combination with the control of the duty ratio D and that of the peak current IH for pulse drive current I within a speckle reduction region Ss in which the variation in the brightness of an image of a given observation target as shown in FIG. 14 is equal to or smaller than a threshold variation, instead of the multi-oscillation mode region Ms in which the wavelength spectrum width W is equal to or larger than the threshold wavelength width Ws.

An index representing the variation in brightness is, for example, speckle contrast. The speckle contrast is defined by a ratio of a standard deviation of the brightness of an image of the observation target to an average value of the brightness. The speckle contrast in the speckle reduction region Ss is, for example, 0.11 or lower. If the speckle contrast is 0.1 or lower, speckles are sufficiently reduced.

As the wavelength spectrum width W increases, the coherence of laser light becomes lower and the speckles become harder to generate. Accordingly, the speckle contrast is lowered. The speckle contrast is in inverse proportion to the wavelength width of laser light emitted from the LD.

A method for measuring the speckle reduction region Ss is the same as a method for measuring the multi-oscillation mode region Ms.

If speckle contrast is measured when the peak current IH of pulse drive current I and the duty ratio are varied for an LD in advance, the speckle reduction region Ss can be grasped from the chart showing a relationship between the peak current IH and the duty ration D as shown in FIG. 14.

A method for setting a route and a light control table 17b at the time of light control in the speckle reduction region Ss is the same as a setting method in the multi-oscillation mode region Ms.

For example, FIG. 15 shows a route from the maximum light intensity state Eb to the minimum light intensity state Ea in the multi-oscillation mode region Ms when the light control is made mainly by controlling the peak current IH of pulse drive current I by the light control circuit 17 (pulse amplitude light control) in the speckle reduction region Ss.

First, light control is made by controlling the peak current IH of pulse drive current I (pulse amplitude light control) from the maximum light intensity state Eb to the multi-oscillation mode threshold current Is (P1 state) in which the duty ratio D is 99%.

Next, in the duty ratio D of the minimum light intensity state Ea, the peak current IH of pulse drive current I is set (P2 state) such that the light intensity is the same as that in the P1 state.

Next, the duty ratio D of pulse drive current I is controlled (pulse amplitude light control) from the P2 state to the minimum light intensity state Ea.

Additional advantages and modifications will readily occur to those skilled in the art. Therefore, the invention in its broader aspects is not limited to the specific details and representative embodiments shown and described herein.

Accordingly, various modifications may be made without departing from the spirit or scope of the general inventive concept as defined by the appended claims and their equivalents.

What is claimed is:

1. An illumination apparatus comprising:
at least one laser diode;
an illumination section which uses light emitted from the laser diode as illumination light; and
a light control circuit which controls light intensity of the laser diode by pulse-modulating a drive current supplied to the laser diode,
wherein that the light control circuit controls the light intensity of the laser diode in combination with a duty ratio and a peak current of the pulse-modulated pulse drive current in a multi-oscillation mode region in which a wavelength spectrum width of the light emitted from the laser diode is equal to or larger than a threshold wavelength width.

2. The illumination apparatus according to claim 1, wherein the light control circuit includes a storage circuit which stores light control information about setting of the duty ratio and the peak current in the multi-oscillation mode region.

3. The illumination apparatus according to claim 2, wherein
when the multi-oscillation mode region includes the peak current corresponding to the duty ratio set in accordance with the light control information, a minimum peak current in the multi-oscillation mode region is defined as a multi-oscillation mode threshold current;
when the multi-oscillation mode region includes the duty ratio corresponding to the peak current set in accordance with the light control information, a minimum duty ratio in the multi-oscillation mode region is defined as a multi-oscillation mode threshold duty ratio; and
the light control circuit controls the light intensity by combining control based on the peak current that is equal to or larger than the multi-oscillation mode threshold current and control based on the duty ratio that is equal to or higher than the multi-oscillation mode threshold duty ratio in the multi-oscillation mode region.

4. The illumination apparatus according to claim 3, wherein the light control circuit controls the light intensity by defining a minimum product of the peak current and the duty ratio as a minimum light intensity state of the illumination light in the multi-oscillation mode region.

5. The illumination apparatus according to claim 1, wherein the threshold wavelength width is set based on a maximum wavelength spectrum width of the light emitted from the laser diode when the illumination light is in a maximum light intensity state.

6. The illumination apparatus according to claim 5, wherein the threshold wavelength width is a wavelength spectrum width that is 70% or larger of the maximum wavelength spectrum width.

7. An illumination apparatus comprising:
at least one laser diode;
an illumination section which uses light emitted from the laser diode as illumination light; and
a light control circuit which controls light intensity of the light emitted from the laser diode by pulse-modulating a drive current supplied to the laser diode,
wherein the light control circuit controls the light intensity of the laser diode in combination with a duty ratio and a peak current of the pulse-modulated pulse drive current in a speckle reduction region in which a variation in brightness caused when an observation target is irradiated with the illumination light is equal to or smaller than a threshold value.

8. The illumination apparatus according to claim 7, wherein the light control circuit includes a storage circuit which stores light control information about setting of the duty ratio and the peak current in the speckle reduction region.

9. The illumination apparatus according to claim 8, wherein:
when the speckle reduction region includes the peak current corresponding to the duty ratio set in accordance with the light control information, a minimum peak current in the speckle reduction region is defined as a speckle reduction region threshold current;
when the speckle reduction region includes the duty ratio corresponding to the peak current set in accordance with the light control information, a minimum duty ratio in the speckle reduction region is defined as a speckle reduction region threshold duty ratio; and
the light control circuit controls the light intensity by combining control based on the peak current that is equal to or larger than the speckle reduction region threshold current and control based on the duty ratio that is equal to or higher than the speckle reduction region threshold duty ratio in the speckle reduction region.

10. The illumination apparatus according to claim 9, wherein the light control circuit controls the light intensity by defining a minimum product of the peak current and the duty ratio as a minimum light intensity state of the illumination light in the speckle reduction region.

11. The illumination apparatus according to claim 9, comprising an image acquiring section which acquires an image of the observation target,
wherein an index representing the variation in brightness falls within a predetermined numerical value including speckle contrast of 0.1 defined by a ratio of a standard deviation of brightness of the image of the observation target to an average value of the brightness.

12. The illumination apparatus according to claim 2, comprising:
an input circuit to which first light intensity control information is to be input to control a light intensity of the illumination light;
an image acquiring section which acquires an image of an observation target; and
an image processor which calculates second light intensity control information based on brightness information of the image of the observation target acquired by the image acquiring section,
wherein the storage circuit stores information of a correlation ratio of the peak current and the duty ratio to the first light intensity control information input from the input circuit or the second light intensity control information calculated by the image processor as the light control information.

13. The illumination apparatus according to claim 8, comprising:
an input circuit to which first light intensity control information is to be input to control a light intensity of the illumination light;
an image acquiring section which acquires an image of the observation target; and
an image processor which calculates second light intensity control information based on brightness information of the image of the observation target acquired by the image acquiring section,
wherein the storage circuit stores information of a correlation ratio of the peak current and the duty ratio to the first light intensity control information input from the input circuit or the second light intensity control information calculated by the image processor as the light control information.

14. The illumination apparatus according to claim 12, comprising:
a plurality of laser diodes which emit lights of different wavelengths;
an optical coupler which combines the lights emitted from the laser diodes,
wherein:
the storage circuit stores the first or second light intensity control information and light intensity ratio information indicating a light intensity ratio of the lights emitted from the laser diodes to cause the illumination light to have a desired color; and
the light control circuit calculates a light intensity necessary for the lights emitted from the laser diodes based on the light intensity ratio information and the first or second light intensity control information, and controls the laser diodes in combination with the peak current and the duty ratio for the laser diodes on the basis of the light control information stored in the storage circuit.

15. The illumination apparatus according to claim 13, comprising:
a plurality of laser diodes which emit lights of different wavelengths;
an optical coupler which combines the lights emitted from the laser diodes,
wherein:
the storage circuit stores the first or second light intensity control information and light intensity ratio information indicating a light intensity ratio of the lights emitted from the laser diodes to cause the illumination light to have a desired color; and
the light control circuit calculates a light intensity necessary for the lights emitted from the laser diodes based on the light intensity ratio information and the first or second light intensity control information, and controls the laser diodes in combination with the peak current and the duty ratio for the laser diodes on the basis of the light control information stored in the storage circuit.

16. The illumination apparatus according to claim 1, wherein the light control circuit sets a bottom current of the pulse drive current obtained by the pulse modulation is set to a value that is equal to or smaller than a lasing threshold value of the laser diode.

17. The illumination apparatus according to claim 7, wherein the light control circuit sets a bottom current of the pulse drive current obtained by the pulse modulation is set to a value that is equal to or smaller than a lasing threshold value of the laser diode.

18. The illumination apparatus according to claim 1, wherein the illumination section includes an optical diffuser which diffuses light emitted from the laser diode and outputs the light diffused by the optical diffuser as the illumination light.

19. The illumination apparatus according to claim 7, wherein the illumination section includes an optical diffuser which diffuses light emitted from the laser diode and outputs the light diffused by the optical diffuser as the illumination light.

20. An endoscope including an illumination apparatus, comprising:
the illumination apparatus of claim 1; and
an image sensor which images an observation target,
when the light control circuit sets a frequency of the pulse drive current obtained by the pulse modulation to an integral multiple which is larger than two for a frame rate of the image sensor.

21. An endoscope including an illumination apparatus, comprising:
the illumination apparatus of claim 7; and
an image sensor which images the observation target,
when the light control circuit sets a frequency of the pulse drive current obtained by the pulse modulation to an integral multiple which is larger than two for a frame rate of the image sensor.
